(19) United States
(12) Patent Application Publication
(10) Pub. No.: US 2015/0045434 A1
(43) Pub. Date: Feb. 12, 2015

(54) METHIONINE COMPOUND INTENDED FOR ANIMAL FEED

(71) Applicant: ROQUETTE FRERES, LESTREM (FR)

(72) Inventor: Guillaume Fiey, Lille (FR)

(73) Assignee: ROQUETTE FRERES, Lestrem (FR)

(21) Appl. No.: 14/363,078

(22) PCT Filed: Dec. 7, 2012
(86) PCT No.: PCT/FR2012/052843

§ 371 Date: Jun. 5, 2014

(30) Foreign Application Priority Data
Dec. 8, 2011 (FR) 1161336

(51) Int. Cl. C07C 319/28; A23K 1/16; C07C 323/58

(52) U.S. Cl. CPC C07C 319/28; C07C 323/58; A23K 1/1634
USPC 514/562

(57) ABSTRACT

The present invention relates to a novel liquid methionine composition, originating from the mother liquor from crystallization of methionine produced by fermentation, comprising between 30 and 50% by weight of methionine and having a dry matter comprised between 20% and 75% by weight and characterized in that it also comprises less than 0.5% by weight of isoleucine and between 0.9% and 1.3% of N-acetyl-methionine.

The present invention relates to a novel liquid methionine composition, derived from the mother liquor from the crystallization of methionine produced by fermentation.

Methionine, like other sulfur amino acids, is essential for cell metabolism. Methionine, however, is not produced by animals, who must then find sufficient quantities in their diet.

It is produced on an industrial scale to be added as a food supplement, in particular in animal feed. Methionine can also be used as a medicament in the treatment or the prevention of various diseases such as allergies or rheumatic fever.

The usual sources of methionine are either proteins of animal origin, or chemical synthesis. However, the decrease in the use of animal proteins following the development of bovine spongiform encephalopathy (BSE), or bird flu, has led to an increase in the demand for synthetic methionine.

D,L-methionine is generally produced from fossil fuels and petrochemical derivatives, in particular from acrolein, methyl mercaptan and cyanides. Obtaining the more active L enantiomer requires additional steps of resolution of racemates which drastically increases production costs.

Today, the production of methionine by biotransformation is an advantageous alternative to petrochemicals due to the depletion of fossil resources and the rising cost of raw materials.

However, the implementation of these processes requires the availability of suitable microorganisms for producing methionine by fermentation of a carbon source.

The first industrially efficient solutions have been published, and in particular described in patent applications WO 2005/111202, WO 2007/017710, WO 2007/077041 and WO 2009/043803.

Other microorganisms that produce methionine are also described in patent applications WO 2004/038013, WO 2006/001616, WO 2006/138689 and WO 2007/012078, in particular.

However, the large-scale production of biosynthetic methionine encounters problems specific to the recovery of chemical molecules in a fermenter, especially for the purification of finished products. In this case, the quality of the resulting crude mixture, the content in impurities and their nature are of major importance.

The present invention therefore relates to a novel liquid methionine composition the methionine content of which makes it directly usable in animal feed.

The methionine composition of the invention is derived from the crystallization mother liquor of methionine produced by fermentation.

This fermentation is conventionally carried out by microorganisms grown on a suitable culture medium comprising a carbon source.

The carbon sources are selected from all carbon sources that can be metabolized by a microorganism, and in particular glucose, sucrose, monosaccharides or oligosaccharides, starch and its derivatives and mixtures thereof.

The composition according to the invention may be distinguished from methionine compositions obtained by other processes by the nature and/or content of the impurities that are present.

The methionine composition according to the invention, derived from the crystallization mother liquor of methionine produced by fermentation, comprises from 30% to 50% by weight of methionine and has a dry matter content comprised between 20% and 75% by weight.

In the spirit of the invention, “a composition having a dry matter content comprised between 20% and 75% by weight” means that said composition has a percentage of dry matter comprised between 20% and 75% by weight relative to the total weight of said composition.

A composition comprises from 30% to 50% by weight of methionine means that said composition comprises methionine in an amount from 30% to 50% by weight relative to the total weight of dry residue (that is to say dry matter) of said composition. Unless otherwise indicated, the percentages herein are given by weight relative to the total weight of the dry residues of the composition. In particular, the percentages of isoleucine, methionine, amino acids other than methionine and isoleucine, or N-acetyl-methionine are expressed by weight relative to the total dry residue weight of the composition.

The composition according to the invention is a composition which typically comprises other residues originating from the process of fermentation of methionine and in particular other amino acids.

The composition according to the invention thus comprises less than 0.5% by weight of isoleucine.

The content of amino acids other than methionine and isoleucine, is advantageously comprised between 7 and 10% by weight.

The content of N-acetyl-methionine is comprised between 0.9 and 1.3% by weight.

The composition according to the invention also and advantageously comprises less than 5% by weight of sugar.

The methionine composition of the invention may be prepared by a process comprising the following steps from the fermentation medium of a methionine-producing microorganism:

1) clarification of the fermentation medium and removal of insoluble and soluble organic impurities from said fermentation medium,

2) optionally, demineralization of the clarified fermentation medium to remove cations and anions from said fermentation medium,

3) crystallization of methionine from the liquid solution thus obtained, and recovery of the crystallization mother liquor,

4) adjustment of the pH of the crystallization mother liquor so as to obtain a value of pH<pKa1 of methionine or a value of pH>pKa2 of methionine,

5) optionally, filtration, and concentration of the mother liquor so treated,

6) recovery of the methionine composition obtained.

The Applicant would like to underline here that the first three steps of this process are common to those it has already described in its international patent application WO 2011/045377, the content of which is incorporated herein by reference.

The first step of the process for preparing the methionine composition according to the invention thus consists in clarifying the fermentation medium and removing the insoluble and soluble organic impurities from said fermentation medium.

In the spirit of the invention, “insoluble organic impurities” is understood to mean the residual insoluble particles, proteins and biomass.

“Soluble organic impurities” designates all soluble particles contaminating the fermentation medium, particularly macromolecules such as soluble proteins and polysaccharides.

The methionine composition of the invention may be obtained by any process of fermentation of methionine with culture of a microorganism optimized to promote the synthesis of methionine, whether it be a bacterium, yeasts or fungi (molds).

Advantageously, the microorganism is selected from Enterobacteriaceae, Bacillaceae, Streptomycetaceae and Corynebacteriaceae.

More particularly, the microorganism is a species selected from the species Escherichia, Klebsiella, Pantoea, Salmonella or Corynebacterium.

Even more particularly, the microorganism is selected from the species Escherichia coli or Corynebacterium glutamicum.

In a preferred embodiment of the invention, the methionine composition according to the invention is derived from the culture of microorganisms described in patent application WO 2009/043803 which is incorporated herein by reference, and more particularly the microorganisms described in the examples. One may, for example, implement the invention using the Escherichia coli strain with genotype MG1655 metA*11 Ptrc-metH PtrcF-cysPUWAM PtrcF-cysJIH Ptrc09-gcvTHP Ptrc36-ARNmst17-metF ΔmetJ ΔpykF ΔpykA ΔpurU (pME101-thrA*1-cysE-PgapA-metA*11) (pCClBAC-serB-serA-serC).

Clarification of the medium is then carried out by any method known to one skilled in the art, for example a method selected from the group consisting of flocculation, sedimentation, membrane technologies (microfiltration, ultrafiltration, nanofiltration and reverse osmosis) and centrifugation.

Removal of soluble organic impurities is carried out by any method known as such by one skilled in the art, for example a method selected from the group consisting of ultrafiltration, heat treatment, treatment with an adsorbant of the activate charcoal type and enzymatic hydrolysis.

The second step of the process for preparing the methionine composition according to the invention, which may be optionally implemented here, consists in then demineralizing said clarified fermentation medium so as to remove cations and anions from said fermentation medium.

This step may be, in this case, performed by conventional electrodialysis or EDC (EURODIA®) and/or by treatment on H+ cation exchange resin (PUROLITE® C120, PUROLITE® C150, PUROLITE® C160 . . . ) and/or anion exchange resin (LEWATIT® S4228, LEWATIT® S4528, Rohm & Haas FPA91 . . . ).

Treatment with ion exchange resins will be preferred to EDC for reasons of cost and efficiency of reduction of salts.

The third step of the process for preparing the methionine composition according to the invention consists lastly in crystallizing the methionine so as to recover methionine in solid form, but above all, within the meaning of the invention, to recover and recycle the crystallization mother liquor.

This crystallization step can be carried out by a technology selected from the group consisting of crystallization by cooling, crystallization by evaporative crystallization and adiabatic crystallization.

The Applicant recommends using evaporative cristallization.

If evaporative crystallization is chosen, the Applicant recommends pre-concentrating the methionine solution by vacuum evaporation with the aid of a falling film evaporator so as to approach supersaturation.

The pre-concentrated solution is then transferred to a crystallizer of the Draft tube type, for example, to be further concentrated and to crystallize.

The solubility of methionine at 35° C. is about 70 g/l. By concentrating the solution to about 250 g/l in a vacuum at a temperature of 35° C., the recovery yield of methionine is >70%.

Conventionally, in crystallization processes, the mother liquor itself is concentrated and recycled into said crystallization process in order to increase the yield of crystallization.

Thus, in its own international patent application WO 2011/045377, the Applicant company itself, noting that the crystallization mother liquor still contained about 40% by weight of methionine relative to the total weight of dry residue, then recommended to optimize the overall yield of crystallized methionine, by recycling the mother liquor upstream of the process, in whole or in part, in liquid form or after a second crystallization jet, before or after a suitable treatment.

The Applicant is therefore going against this technical bias by now choosing to maximize said mother liquor, not as a byproduct to be recycled, but as the direct source of a methionine composition with added value.

The process for preparing the methionine composition according to the invention therefore consists in adding to the process the last three additional following steps.

The fourth step consists in adjusting the pH of the crystallization mother liquor so as to situate at a value of pH<pKa1 of methionine or at a value of pH>pKa2 of methionine.

In a first preferred embodiment of the process according to the invention, the pH of the crystallization mother liquor is adjusted so as to situate at a pH<pKa1 (pKa1=2.2) of methionine by acidification of the mother liquor.

This acidification is carried out by any method known by one skilled in the art.

The Applicant recommends acidification with 37% hydrochloric acid to a pH value less than 2.2 (pKa of the acid function of methionine).

In a second preferred embodiment of the process according to the invention, the pH of the crystallization mother liquor is adjusted so as to situate at a pH>pKa2 of methionine by alkalinization of the mother liquor.

The Applicant recommends alkalinization with 50% sodium hydroxide to a pH value greater than 9.3 (pKa of the amine function of methionine).

The fifth step may consist in filtering the solution to remove a precipitate composed notably of xanthine, and then to concentrate the mother liquor thus treated.

According to the first preferred embodiment, the acidified solution is then filtered through a membrane with porosity 5 μm, and concentrated to obtain a dry matter percentage comprised between 20% and 75% by weight.

According to the second preferred embodiment, the alkalinized solution is filtered and concentrated to obtain a dry matter percentage comprised between 20% and 75% by weight.

The sixth step is to recover the liquid methionine composition according to the invention.

The composition according to the invention can advantageously be employed directly in animal feed as a supplement or food additive supplied to animals, mixed with the diet provided to each animal, as premix, in the form of a premixed or extemporaneously mixed composition, or independently of other foods.

The invention therefore also relates to a food additive comprising the methionine composition according to the invention, preferably intended for animal feed.

One skilled in the art knows the amounts of methionine required for animal feed in a diet suitable to each animal and will therefore determine how to use the composition according to the invention and in what quantity.

In particular, the composition according to the invention is particularly suitable thanks to its supply of trace elements and water to facilitate the dosing, mixing and hydration of the usual food of the animal.

Other features and advantages of the invention will become apparent upon reading the following examples. They are, however, given for purposes of illustration and not by way of limitation.

### EXAMPLE

A methionine-producing strain of Escherichia coli with genotype MG1655 metA*11 Ptrc-metH PtrcF-cysPUWAM PtrcF-cysJIH Ptrc09-gcvTHP Ptrc36-ARNmst17-metF ΔmetJ ΔpykF ΔpykA ΔpurU (pME101 -thrA*1-cysE-PgapA-metA*11) (pCClBAC-serB-serA-serC), described in patent application WO 2009/043803, is grown in fermentation culture conditions according to the method described in this same patent application.

The fermentation broth resulting from the implementation of said strain is purified as follows.

### A) Removal of Insoluble Organic Impurities (Biomass)

Elimination is carried out by tangential filtration on a membrane having a pore diameter of 100 nm, between 40 and 80° C. (ceramic type membrane with 3.5 mm channel diameter).

Preferably the temperature is maintained at 40° C. with a transmembrane pressure of 1 bar and a diafiltration with 20% deionized water.

Under these conditions, the average flow is 30 l/h/m² and the permeate obtained is clear and bright.

The permeate, free of biomass and insoluble particles, still contains soluble organic impurities, particularly sugars and soluble proteins that should be eliminated before crystallization.

### B) Removal of Soluble Organic Impurities (Sugars and Soluble Macromolecules)

This step aims to eliminate sugars (polysaccharides) and macromolecules contained in the fermentation broth.

This elimination is carried out by ultrafiltration on a ceramic membrane with a cutoff threshold of 5 kDa.

At 40° C., the average filtration flow is 25 l/h/m² and about 70% of the macromolecules are held back in the retentate.

### D) Crystallization

The above solution is pre-concentrated by evaporation of water at 50° C. on a falling film vacuum evaporator of the WIEGAND® type.

The concentration factor is on the order of 2 to 5 according to the initial concentration of L-methionine.

Here it is equal to 3 to be closer to supersaturation at 50° C. (80 g/l).

The pre-concentrated solution is then transferred to a forced circulation evaporative crystallizer to be further concentrated and crystallized under vacuum (50 mBar) at about 35° C.

The concentration factor applied in this evaporative crystallizer is about 3, so as to reach 240 g/l.

After separation on a ROUSSELET® centrifuge with polypropylene fabric (120 m³/m²/h) and washing with one volume of deionized water per volume of cake, the crystals are dried in a fluidized bed at 45° C. (AEROMATIC® type).

Under these conditions the recovery yield of L-methionine is >80% for purity >85%/dry.

The crystallization mother liquor has a composition described in Table 1 below:

TABLE 1

| | Composition of crystallization |
|---|---|
| g/100 g of dry residue | mother liquor |
| L-Methionine (L-MET) | 30-70 |
| N-acetyl-Methionine (NAM) | 1.3-2 |
| Isoleucine | <0.5 |
| Cations (except NH₄⁺) | 2-7 |
| Anions (except Cl⁻) | 1-20 |
| Cl⁻ | 0-1 |
| NH₄⁺ | 0.5-10 |
| Assay protein nitrogen | 0.1-1.5 |
| N 6.25 | |
| Other amino acids | 5-40 |
| Sugars (Glucose . . .) | 0.5-5 |

The mother liquor here contained even more than 30% methionine dry weight.

It was then chosen to acidify, filter and concentrate the mother liquor:

1) acidification by addition of 37% hydrochloric acid to a pH of around 1.6,

2) filtration on membrane with 5 μm porosity,

3) concentration in a laboratory Rotavapor (water bath 80° C., under vacuum of 50 mbar and vapor temperature of 35° C.).

This results in a composition of mother liquor according to the invention with a dry matter content of 60%, as shown in the following Table 2.

TABLE 2

| | Composition of crystallization |
|---|---|
| g/100 g of dry residue | mother liquor |
| L-Methionine (L-MET) | 30-50 |
| N-acetyl-Methionine (NAM) | 0.9-1.3 |
| Isoleucine (ISO) | <0.5 |
| Cations (except NH₄⁺) | 1-6 |
| Anions (except Cl⁻) | 1-20 |
| Cl⁻ | 10-25 |
| NH₄⁺ | 0.2-8 |
| Assay protein nitrogen | 0.1-1.5% |
| N 6.25 | |
| Other amino acids | 5-40 |
| Sugars (Glucose . . .) | 0.2-5 |

This methionine composition, despite being relatively rich in chlorides, is quite usable as animal feed, allowing supplementation of the food in methionine to obtain compositions classically comprising up to 0.5% added methionine.

1-7. (canceled)

8. A liquid methionine composition, derived from the mother liquor of crystallization of methionine produced by fermentation which comprises from 30% to 50% by weight of methionine, and which has a dry matter content between 20% and 75% by weight.

9. The composition of claim 8, which comprises less than 0.5% by weight of isoleucine and between 0.9% and 1.3% by weight of N-acetyl-methionine.

10. The composition of claim 8, wherein the content of amino acids other than methionine and isoleucine is between 7% and 10% by weight.

11. The composition of claim 8, wherein the sugar content is less than 5%.

12. A process for preparing a composition of claim 8, which comprises the following steps from the fermentation medium of a methionine-producing microorganism:
a) clarification of the fermentation medium and removal of insoluble and soluble organic impurities from said fermentation medium,
b) optionally, demineralization of the clarified fermentation medium to remove cations and anions from said fermentation medium,
c) crystallization of methionine from the liquid solution thus obtained, and recovery of the crystallization mother liquor,
d) adjustment of the pH of the crystallization mother liquor so as to situate at a value of pH<pKa1 of methionine or a value of pH>pKa2 of methionine,
e) optionally, filtration, and concentration of said solution, and
f) recovery of the methionine composition thus obtained.

13. A method for preparing a food additive or a supplement for animal feed which comprises mixing the composition of claim 8 with another food.

14. A method for preparing a food additive or a supplement for animal feed which comprises mixing a composition obtained by the process of claim 12 with another food.

15. A food additive for animal feed comprising a composition according to claim 8 and an animal food.

16. A food additive for animal feed comprising a composition obtained by the process of claim 12 and an animal food.
